(19) United States
(12) Patent Application Publication
(10) Pub. No.: US 2026/0192120 A1
(43) Pub. Date: Jul. 9, 2026

(54) ACCELERATED MAGNETIC STIMULATION PROCEDURE AND DEVICE

(71) Applicant: Neuronetics, Inc., Malvern, PA (US)

(72) Inventor: Eleanor Cole, Los Angeles, CA (US)

(73) Assignee: Neuronetics, Inc., Malvern, PA (US)

(21) Appl. No.: 19/442,714

(22) Filed: Jan. 7, 2026

Related U.S. Application Data
(60) Provisional application No. 63/742,698, filed on Jan. 7, 2025.

(51) Int. Cl. A61N 2/00; A61N 2/02

(52) U.S. Cl. CPC A61N 2/006; A61N 2/02

(57) ABSTRACT

A pulsing magnetic field related to magnetic stimulation therapy of a patient may be generated. For example, one or more parameters, including a number of pulses of the pulsing magnetic field for one or more treatment session(s), a frequency of the pulsing magnetic field for the treatment session(s), an inter-train interval of the pulsing magnetic field for the treatment session(s), and/or an intensity of the pulsing magnetic field for the treatment session(s) may be determined. The number of pulses may be within a range of 2000 to 4000 pulses. The inter-train interval may be within a range of 11 to 14 seconds. An amount of time between consecutive treatment sessions may be within a range of 5 to 240 minutes.

### CROSS-REFERENCE TO RELATED APPLICATIONS

This application claims the benefit of U.S. Provisional Application No. 63/742,698, filed Jan. 7, 2025, the disclosure of which is incorporated herein by reference in its entirety.

### BACKGROUND

A number of medical ailments may be treated and/or diagnosed through the application of a magnetic field to an afflicted portion of a patient's body. Neurons and muscle cells may be a form of biological circuitry that carry electrical signals and respond to electromagnetic stimuli. When a conductive wire loop is passed through a magnetic field or is in the presence of a changing magnetic field, an electric current may be induced in the wire. The same principle may hold true for conductive biological tissue. When a changing magnetic field is applied to a portion of the body, neurons may be depolarized and stimulated.

A nerve cell or neuron may be stimulated in a number of ways, for example, non-invasively via transcranial magnetic stimulation (TMS). TMS may use a rapidly changing magnetic field to induce a current on a nerve cell, without having to cut or penetrate the skin. The nerve may “fire” when a membrane potential within the nerve rises with respect to its normal negative ambient level of approximately −90 mV, for example, depending on the type of nerve, local pH of the surrounding tissue, and/or peripheral nerve stimulation.

A magnetic stimulation component may be used to produce the rapidly changing magnetic field inducing a current on a nerve cell. The magnetic stimulation component may fail or operate improperly during treatment, which may result in improper treatment for the patient. For example, the magnetic component may appear to operate correctly, but actually may be producing magnetic field pulses outside of designed device specifications, potentially resulting in improper diagnosis and/or therapy being administered to the patient. Administering an incorrect magnetic field pulse to a patient can affect the magnetic stimulation diagnosis and/or treatment adversely. For example, the treatment provider may believe that the patient is not responding to the treatment, when in fact the intended treatment is not being administered to the patient. Thus, the treatment provider and/or diagnosing clinician may be led to make treatment decisions based on faulty information.

### SUMMARY

A method, system, and apparatus for magnetic stimulation therapy may be provided. For example, the magnetic stimulation therapy may include one or more treatment sessions performed according to an accelerated DASH protocol.

A pulsing magnetic field related to magnetic stimulation therapy of a patient may be generated. For example, a plurality of signals associated with the generation of the pulsing magnetic field for a plurality of treatment sessions associated with one or more patients may be generated. Generating the plurality of signals for one or more treatment sessions may include determining one or more of a number of pulses of the pulsing magnetic field for the treatment session(s), a frequency of the pulsing magnetic field for the treatment session(s), an inter-train interval of the pulsing magnetic field for the treatment session(s), and/or an intensity of the pulsing magnetic field for the treatment session(s). The pulsing magnetic field may be generated for the magnetic stimulation therapy in response to the signals based on the determined number of pulses, frequency, inter-train interval, and intensity of the pulsing magnetic field for the treatment session. The number of pulses may be within a range of 1,700 to 6,000 pulses. The inter-train interval may be within a range of 11 to 14 seconds. An amount of time between consecutive treatment sessions may be within a range of 5 to 240 minutes.

The number of pulses may be within a range of 2,800 to 5000 pulses, or more preferably within a range of 3000 to 4000 pulses. For example, the number of pulses may be 3,000 pulses. The inter-train interval may be approximately 11 seconds. The frequency may be within a range of 5 to 20 Hz, or more preferably within a range of 5 to 15 Hz. For example, the frequency may be approximately 10 Hz. The number of pulses for a treatment session may be subdivided into one or more pulse trains, and the inter-train interval may represent an amount of time between generating respective pulse trains. For example, each pulse train may include between 15 and 120 pulses. A treatment session may include between 30 and 120 pulse trains, or more preferably between 50 and 100 pulse trains, or even more preferably between 70 and 90 pulse trains. The duration of each pulse train may be between 1 and 12 seconds, or more preferably between 4 and 8 seconds.

The one or more treatment sessions may include a first treatment session and a second treatment session, which may both be administered to the same patient on the same day. The amount of time between the first treatment session and the second treatment session (e.g., an inter-session interval) may be between 15 and 90 minutes, or more preferably between 30 and 75 minutes, and even more preferably between 45 and 60 minutes. The number of pulses for the first treatment session may be the same as or different from the number of pulses for the second treatment session. The intensity of the magnetic field for the first treatment session may be the same as or different from the intensity of the magnetic field for the second treatment session.

The intensity of the pulsing magnetic field for a treatment session may be determined based on an intensity required to provoke a motor threshold (MT) response in a given patient. For example, the intensity of the pulsing magnetic field may be within a range of 50% to 140% of the intensity required to provoke the MT response in the patient, or more preferably within a range of 110% to 120% of the intensity required to provide the MT response in the patient. For example, the intensity of the magnetic field may be approximately 120% of the intensity required to provoke the MT response in the patient. The intensity required to provoke the MT response in the patient may be determined by determining a location of a reference point (e.g., an MT location) on the patient, determining a first intensity for a first pulse of the pulsing magnetic field based on one or more factors associated with the patient, generating the first pulse of the pulsing magnetic field at the location of the reference point at the first intensity, determining that the first pulse did not provoke the MT response in the patient, determining a second intensity for a second pulse of the pulsing magnetic field, wherein the second intensity is higher than the first intensity, generating the second pulse of the pulsing magnetic field at the location of the reference point at the second intensity, determining that the second pulse provoked the MT response in the patient, and/or setting the second intensity as the intensity required to provoke a motor threshold MT response. Alternatively, the patient may be a repeat patient, and the intensity required to provoke the MT response may include determining an identity of the patient, accessing a database that includes one or more associations between respective patient identities and intensities required to provoke the MT response in the respective patients, and/or determining the intensity required to provoke the MT response in the patient based on the associations in the database.

One or more treatment locations on the patient may be determined. For example, determining the treatment location(s) may include determining a location of an MT location on the patient, measuring a predetermined distance from the MT location, wherein the predetermined distance is within a range of 4.0 and 7.0 cm, and/or determining the treatment location(s) based on measuring the predetermined distance from the MT location. The predetermined distance may be between 5.0 and 6.0 cm. Alternatively, the treatment location(s) may be determined by determining a location of a reference point defined by a support structure supported by a head of the patient, determining a position angle associated with the treatment location relative to the reference point (e.g., where the position angle may represent an angular offset between a line located within a median plane of the patient that intersects with the reference point and a line that intersects the reference point and an MT location of the patient), determining a distance (e.g., an arc length) between the support structure and the treatment location(s), and/or determining the treatment location(s) based on the distance between the support structure and the treatment location and the position angle. The treatment location(s) may be a left dorsolateral prefrontal cortex (DLPFC) of the patient. The treatment location(s) may be determined without requiring the use of magnetic resonance imaging (MRI) data. Alternatively, the treatment location(s) may be determined based on one or more structural or functional features in a medical image.

Determining the treatment location(s) may include measuring a head circumference of the patient, a nasion-inion distance of the patient, and a head length of the patient, determining one or more standardized coordinates associated with the treatment location(s), and/or calculating the treatment location(s) relative to a surface reference location based on the head circumference, the nasion-inion distance, the head length, and the standardized coordinates. For example, calculating the treatment location(s) relative to the surface reference location may include transforming one or more target coordinates to device coordinates, estimating a distance of the treatment location(s) from the surface reference location based on the head circumference, the nasion-inion distance, and the head length, and/or determining an angle relative to one or more of a head feature or a device feature based on the transformed coordinates and the head circumference, the nasion-inion distance, and the head length. The distance of the treatment location(s) from the surface reference location may be estimated based on a ratio of a surface distance to the head length, and the angle may be determined based on the ratio of the surface distance to the head length. The surface reference location may be Brodmann area 46.

Alternatively, the treatment location(s) may be determined based on one or more additional measurements (e.g., an ear-to-ear distance), and the distance of the treatment location(s) from the surface reference location may be estimated based on a ratio of the ear-to-ear distance to a measured head width. For example, the treatment location(s) may be determined by measuring a head circumference of the patient, an ear to ear distance of the patient, and a head width of the patient, determining one or more standardized coordinates associated with the treatment location, and/or calculating the treatment location(s) relative to a surface refence location based on the head circumference, the ear to ear distance, the head width, and the standardized coordinates. The treatment location may be determined based on one or more of an EEG, a heart rate measurement, or a blood oxygenation measurement (e.g., MRI, fMRI, fcMRI, fNIRS).

### BRIEF DESCRIPTION OF THE DRAWINGS

FIG. 1A is a block diagram illustrating an example of a magnetic stimulation system.

FIG. 1B is a diagram of an example of a treatment or diagnostic system.

FIG. 2 is a block diagram illustrating an example of a magnetic stimulation system.

FIG. 3 is a diagram illustrating an example waveform of a signal received by a controller of a magnetic stimulation system.

FIG. 4 is a flow diagram of an example TMS treatment process.

FIG. 5 is a flow diagram of another example TMS treatment process.

FIG. 6 is a flow diagram of an example method for determining a treatment location for a TMS treatment process.

### DETAILED DESCRIPTION

A system may be provided that monitors a pulsing magnetic field related to magnetic stimulation therapy, for example, to determine whether or not a system failure has occurred. The system may comprise a magnetic stimulation component, a sensor, and a processor. The magnetic stimulation component may be configured to generate a pulsing magnetic field for performing magnetic stimulation therapy on a patient. The sensor may be configured to generate a signal associated with the pulsing magnetic field of the magnetic stimulation component. For example, the sensor may be placed between the magnetic stimulation component and the patient. In response to the pulsing magnetic field, a current signal, a voltage signal, or the like may be generated in the sensor that may be proportional to the pulsing magnetic field.

The processor may be configured to estimate one or more characteristics (e.g., two characteristics) associated with the signal (e.g., the generated signal) and determine, based on the estimated characteristics, a characteristic of the signal. The processor may be configured to determine whether a failure has occurred based on the determined characteristic of the signal. As such, the system may be able to detect whether the pulsing magnetic field is properly providing magnetic stimulation therapy to the patient. Upon detecting a failure, the system may be configured to pause the TMS procedure, shut down the magnetic stimulation system, alert a user of the magnetic stimulation system, and/or alter a current applied to the magnetic stimulation component.

In 1831, Michael Faraday discovered that the magnitude of an electric field induced on a conductor is proportional to the rate of change of magnetic flux that cuts across the conductor. Faraday's law, well known to those skilled in the art, may be represented as E~−(A*dB/dt), where E is the induced electric field in volts/meter and dB/dt is the time rate of change of magnetic flux density in Tesla/second. In other words, the amount of electric field induced in an object, such as a conductor, may be determined using two factors: the magnetic flux density and the time rate of change of the flux. The greater the flux density and its derivative, the greater the induced electric field and resulting current density. Magnetic flux may be a function of distance. For example, because the magnetic flux density may decrease in strength with relation to the distance from the source of the magnetic field (e.g., 1/r³, 1/r⁵, or the like), the flux density may be greater the closer the conductor is to the source of the magnetic field. When the conductor is a coil, the current induced in the coil by the electric field may be increased in proportion to the number of turns of the coil.

An overview of an example operation and application of a magnetic system in which aspects of the various embodiments may be implemented may be provided. The magnitude of an electric field induced on a conductor may be proportional to the rate of change of magnetic flux density across the conductor. When an electric field is induced in a conductor, the electric field may create a corresponding current flow in the conductor. The current flow may be in the same direction of the electric field vector at a given point. The peak electric field may occur when the time rate of change of the magnetic flux density is the greatest and may diminish at other times. During a magnetic pulse, the current may flow in a direction that tends to preserve the magnetic field (e.g., Lenz's Law).

Certain parts of the anatomy (e.g., nerves, tissue, muscle, brain) may act as a conductor and may carry electric current when an pulsed magnetic field is applied. The pulsed magnetic field may be applied to these parts of the anatomy non-invasively. For example, in the context of TMS, a time-varying magnetic field may be applied across the skull to create an electric field in the brain tissue, which may produce a current. If the induced current is of sufficient density and/or duration, neuron action potential may be reduced to the extent that the membrane sodium channels open and an action potential response is created. An impulse of current may be propagated along the axon membrane that transmits information to other neurons via modulation of neurotransmitters. Such magnetic stimulation may acutely affect glucose metabolism and local blood flow in cortical tissue. In the case of major depressive disorder, neurotransmitter dysregulation and abnormal glucose metabolism in the prefrontal cortex and the connected limbic structures may be a likely pathophysiology. Repeated application of magnetic stimulation to the prefrontal cortex may produce chronic changes in neurotransmitter concentrations, metabolism, and/or nerve changes to stimulation thresholds, for example, such that depression may be alleviated.

Non-cortical neurons (e.g., cranial nerves, peripheral nerves, sensory nerves) may be stimulated by an induced electric field. For example, peripheral nerves may be intentionally stimulated to diagnose neuropathologies, for example, by observing response times and conduction velocities in response to a pulsed magnetic field induced stimulus. Discomfort and/or pain may result if the induced electric field applied to a peripheral and/or cranial nerve is very intense, and/or focused on a small area of the nerve. This discomfort may be diminished, for example, by intentionally over-stimulating the sensory nerves in the affected nerve bundle so that they can no longer respond to external pain stimuli, or by reducing the intensity and/or focus of the induced electric field that is causing the pain sensation.

Transcutaneous magnetic stimulation may not be limited to treatment of depression. Transcutaneous magnetic stimulation may be used to treat a patient, such as a human, for example, suffering from epilepsy, schizophrenia, Parkinson's disease, Tourette's syndrome, amyotrophic lateral sclerosis (ALS), multiple sclerosis (MS), Alzheimer's disease, attention deficit/hyperactivity disorder, obesity, bipolar disorder/mania, anxiety disorders (e.g., panic disorder with and without agoraphobia, social phobia also known as social anxiety disorder, acute stress disorder and/or generalized anxiety disorder), post-traumatic stress disorder (one of the anxiety disorders in DSM), obsessive compulsive disorder (e.g., one of the anxiety disorders in DSM), pain (such as, for example, migraine and trigeminal neuralgia, as well as chronic pain disorders, including neuropathic pain, e.g., pain due to diabetic neuropathy, post-herpetic neuralgia, and idiopathic pain disorders, e.g., fibromyalgia, regional myofascial pain syndromes), rehabilitation following stroke (neuro plasticity induction), tinnitus, stimulation of implanted neurons to facilitate integration, substance-related disorders (e.g., dependence, abuse and withdrawal diagnoses for alcohol, cocaine, amphetamine, caffeine, nicotine, cannabis and the like), spinal cord injury and regeneration/rehabilitation, stroke, head injury, sleep deprivation reversal, primary sleep disorders (primary insomnia, primary hypersomnia, circadian rhythm sleep disorder), cognitive enhancements, dementias, premenstrual dysphoric disorder (PMS), drug delivery systems (changing the cell membrane permeability to a drug), induction of protein synthesis (induction of transcription and translation), stuttering, aphasia, dysphagia, essential tremor, autism spectrum disorders, and/or eating disorders (such as bulimia, anorexia and binge eating).

A device may take advantage of the above principles to induce an electric field used in a variety of applications. For example, a magnetic device may be used for electrical stimulation of the anatomy. While the discussion herein focuses on magnetic devices that are used in connection with magnetic stimulation of anatomical tissue, a magnetic device may be utilized in any field of endeavor.

A ferromagnetic core may be used in connection with a magnetic device to produce a magnetic field. For example, a ferromagnetic core may include an arc-shaped (e.g., approximately hemispherical) magnetic material. A ferromagnetic core may include a highly saturable magnetic material having a magnetic saturation of at least 0.5 Tesla. A ferromagnetic core may be shaped to optimize the magnetic field distribution in the treatment area. For example, such a magnetic field may be for purposes of carrying out transcutaneous magnetic stimulation such as, for example, Transcranial Magnetic Stimulation (TMS), Repetitive TMS (rTMS), Magnetic Seizure Therapy (MST), deep TMS (dTMS), controlled and/or varied pulse shape TMS (cTMS), reduction of peripheral nerve discomfort, etc. Although examples described herein may be discussed in connection with TMS and rTMS, the examples described herein may be utilized in connection with any type of magnetic stimulation, such as transcutaneous magnetic stimulation, for example. Furthermore, the embodiments presented herein are not limited to the use of ferromagnetic core magnetic stimulation systems, as other core materials may be used such as, for example, an air core.

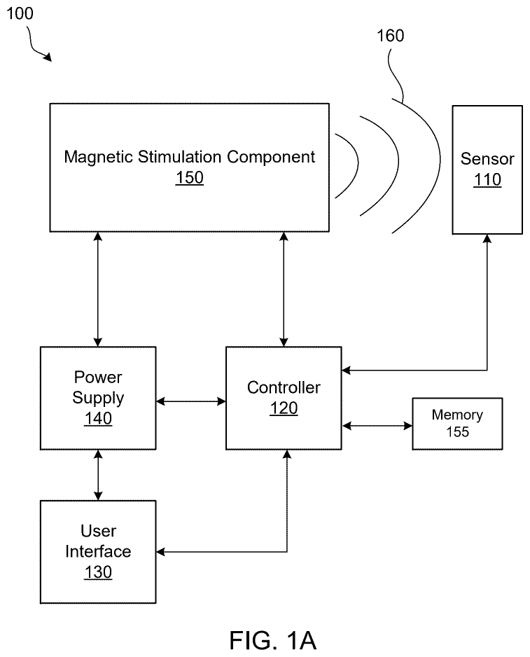

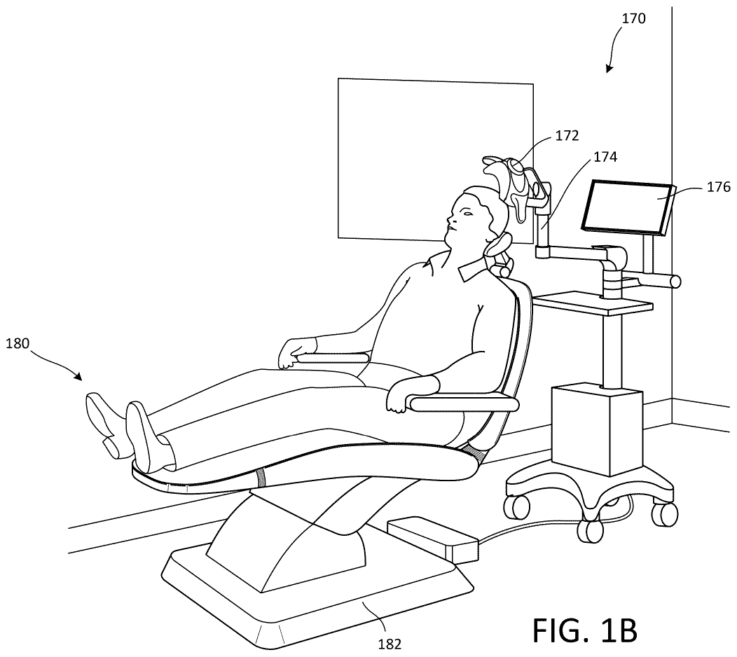

FIG. 1A is a block diagram illustrating an example of a magnetic stimulation system. A magnetic stimulation system 100 may comprise a sensor 110, a controller 120, a user interface 130, a power supply 140, a magnetic stimulation component 150, and a memory 155. A magnetic stimulation device may refer to one or more components of a magnetic stimulation system (e.g., the magnetic stimulation system 100).

The magnetic stimulation component 150 may be configured to generate a pulsing magnetic field 160 to conduct magnetic stimulation therapy on a treatment area of a patient. The magnetic stimulation therapy may be, for example, transcranial magnetic stimulation (TMS). TMS may refer to TMS, repetitive transcranial magnetic stimulation (rTMS), deep TMS (dTMS), cTMS, or the like. The magnetic stimulation component 150 may be a treatment coil. The magnetic stimulation component 150 may include a single treatment coil, multiple treatment coils and/or an array of treatment coils. The treatment area may be the prefrontal cortex, for example. The magnetic stimulation component 150 may or may not include a core, such as a magnetic core (e.g., ferromagnetic core), for example. The pulsing magnetic field 160 may include one or more pulse trains. A pulse train (e.g., each pulse train) of the pulsing magnetic field 160 may include one or more pulses.

The sensor 110 may be configured to generate a signal associated with a pulsing magnetic field 160. The sensor 110 may be placed between the magnetic stimulation component 150 and a treatment area of a patient. The sensor 110 may be configured to generate a signal associated with the pulsing magnetic field 160 of the magnetic stimulation component 150 (e.g., a signal induced by the pulsing magnetic field 160). For example, the sensor 110 may convert a physical property (e.g., the strength of pulsing magnetic field 160) into a corresponding electrical signal (e.g., a current signal or a voltage signal). As such, the sensor 110 may detect and/or measure a physical parameter of the pulsing magnetic field and generate a signal associated with the pulsing magnetic field using the detected/measured physical parameter. The generated signal may be a voltage signal, a current signal, and/or the like that may be proportional to a change in the pulsing magnetic field 160. For example, a current may be generated in the sensor 110 that may be proportional to the pulsing magnetic field 160. The sensor 110 may generate a voltage that may be proportional to the magnetic flux density (dB/dt) of the pulsing magnetic field 160.

The sensor 110 may include one or more of a conductive coil, a loop (e.g., having a number of turns based on the pulsing magnetic field), a Hall sensor, a magnetoresistive material, a Faraday effect sensor, a Kerr effect sensor, a flux gate sensor, an inductance change element, a nerve tissue response measurement device, an electric field sensor (e.g., in a conductive field), and/or the like. The sensor 110 may be configured to generate more than one signal, for example, more than one signal that is associated with the pulsing magnetic field 160 generated by the magnetic stimulation component 150.

The controller 120 may be any type of hardware, software, or combination thereof. The controller 120 may be configured to control one or more of the components of the magnetic stimulation system 100, such as the sensor 110, the user interface 130, the power supply 140, and/or the magnetic stimulation component 150, for example to conduct magnetic stimulation therapy. For example, the controller 120 may include a general purpose processor, a special purpose processor, a conventional processor, a digital signal processor (DSP), a plurality of microprocessors, one or more microprocessors in association with a DSP core, a microcontroller, any other type of integrated circuit (IC), a state machine, and/or the like.

The controller 120 may be configured to receive inputs from the user interface 130 and/or the sensor 110 to conduct magnetic stimulation therapy accordingly. For example, the controller 120 may perform signal coding, data processing, power control, input/output processing, and/or any other functionality that enables the controller 120 to operate the magnetic stimulation component for magnetic stimulation. Although the controller 120 of the magnetic stimulation system 100 may be configured to control both the magnetic stimulation component 150 and the sensor, the magnetic stimulation system 100 may include two or more controllers for individually controlling two or more of the components of the magnetic stimulation system 100.

The controller 120 may be configured to estimate (e.g., measure) characteristics associated with the signal generated by the sensor 110 (e.g., associated with one or more peaks of the signal). The controller 120 may estimate a subset of the pulses of the signal or may estimate the signal continuously. By estimating characteristics of the signal, the controller 120 may estimate a model of what is incurring in the brain of a patient in response to the pulsing magnetic field.

As described herein, the controller 120 may be configured to estimate a characteristics associated with the signal generated by the sensor 110. For example, the controller 120 may estimate a voltage associated with the generated signal (e.g., a voltage associated with a peak of the generated signal), a current associated with the generated signal (e.g., a current associated with a peak of the generated signal), a Root Mean Square (RMS) value associated with the generated signal (e.g., a RMS value associated with a peak of the generated signal), a zero-cross time associated with the generated signal (e.g., a zero-cross time associated with a peak of the generated signal), a time value associated with the generated signal (e.g., a time value associated with a peak of the generated signal), and/or a time duration of the generated signal (e.g., a time duration associated with a peak of the generated signal, for example, peak duration). For example, the peak duration may be characterized by a time duration between an initial rising edge of a peak and a first zero-crossing of the peak.

The controller 120 may determine one or more characteristics associated with the signal generated by the sensor 110. For example, the controller 120 may determine characteristic(s) associated with the signal using one or more estimated characteristics of the signal. As described herein, the controller 120 may determine a decay rate of the generated signal, a frequency of the generated signal, a timing associated with the generated signal, a magnetic flux associated with the generated signal, a pulse shape of the generated signal, a voltage associated with the generated signal, and/or a current associated with the generated signal.

The pulsing magnetic field 160 may be characterized by one or more pulse trains. A pulse train may be characterized by one or more pulses. Consecutive pulse trains may be separated by a time interval (e.g., an inter-train interval). Characteristics associated with the signal generated by the sensor 110 may be associated with the same pulse of the pulsing magnetic field 160 or different pulses of the pulsing magnetic field 160. The characteristics associated with the generated signal may be associated with the same pulse train of the pulsing magnetic field 160 or different pulse trains of the pulsing magnetic field 160.

The controller 120 may determine whether a failure has occurred based on one or more characteristics of the signal generated by the sensor 110. For example, the controller 120 may determine whether a failure has occurred based on whether a characteristic of the generated signal (e.g., a determined characteristic) is outside of a predetermined acceptance window. The predetermined acceptance window may be defined based on an expected signal associated with the pulsing magnetic field. The acceptance window may be adjustable based on the settings of the type of magnetic stimulation therapy, the magnetic stimulation treatment parameters, and/or the patient parameters. For example, the controller 120 may receive settings of the magnetic stimulation procedure and compare the settings of the magnetic stimulation procedure with characteristic(s) of the generated signal to determine whether a failure has occurred. For example, the controller 120 may determine that a failure has occurred when a difference between two or more characteristics (e.g., characteristics relating to peaks) of the generated signal exceeds an expected value (e.g., which may be determined based on magnetic stimulation settings). For example, the expected value may include an expected voltage value, an expected current value, and/or the like.

In the event a failure is determined to have occurred, the controller 120 may enter a failure mode. In the failure mode, the controller 120 may pause the magnetic stimulation procedure, shut down the magnetic stimulation component 150, alert a user of the magnetic stimulation system 100, and/or alter a current applied to the magnetic stimulation component 150. For example, when the controller 120 enters the failure mode, the controller 120 may adjust the frequency at which it estimates characteristics of the generated signal. For example, the controller 120 may check for failures more frequently after a first failure is detected.

The controller 120 may log one or more characteristics of the signal generated by the sensor 110 (e.g., estimated characteristics and/or determined characteristics). The controller 120 may be configured to log one or more failures of the magnetic stimulation procedure. In one or more embodiments, the magnetic stimulation system 100 may include an indicator that may indicate to a user of the magnetic stimulation system 100 that a failure has occurred. For example, the indicator may be a light, a speaker, an icon displayed on the user interface 130, and/or the like.

The user interface 130 may be any type of interface in which a user of the magnetic stimulation system 100 may initiate, adjust, and/or end the magnetic stimulation procedure. For example, the user interface may include a personal computer (PC), a keyboard, a mouse, a touchscreen, a wireless device, and/or the like, that allows for an interface between the user and the magnetic stimulation system 100.

The power supply 140 may be any type of power source that provides sufficient energy for the magnetic stimulation component 150 to generate the pulsing magnetic field 160 for its intended purpose, for example, for TMS, rTMS, MST or any other type of application. For example, the power supply 140 may be a conventional 120 or 240 VAC main power source.

FIG. 1B is a diagram of an example of a treatment or diagnostic system 170. The treatment or diagnostic system 170 may comprise a processor (not shown), a power supply (not shown), memory (not shown), a transceiver, (not shown), a treatment coil 172 (e.g., stimulation coil), an articulating arm 174, a display device 176, a human subject positioning apparatus 182, and/or a motion detection device, such as a motor threshold detection device.

The treatment system 170 may be stationary or movable. For example, the treatment system 170 may be integrated into a movable cart, for example, as shown in FIG. 1B. In one or more examples, the treatment system 170 may be a TMS treatment system (e.g., NeuroStar®) and/or any other therapeutic and/or diagnostic procedure system.

The treatment coil 172 (e.g., an electromagnet) may be used to administer a therapeutic and/or diagnostic procedure to a human subject 180, for example, TMS. Example treatment coils 172 may include one or more treatment coils and one or more ferromagnetic components that are configured to be disposed proximate to corresponding ones of the one or more treatment coils. The one or more treatment coils and ferromagnetic components of each TMS device may cooperatively generate a magnetic field that exhibits one or more characteristics that differ from those of a magnetic field that is generated by the one or more treatment coils alone. For example, the treatment system 170 may include a drive circuit (not shown) that may be configured to cause the treatment coil 172 to generate a magnetic field. Examples of a drive circuit that may be used in the treatment system 170 are described in U.S. Pat. No. 7,744,523, which is hereby incorporated by reference in its entirety. The processor of the treatment system 170 may be configured to generate one or more drive signals (e.g., via the drive circuit) that are configured to cause the treatment coil 172 to generate a pulsating magnetic field. The pulsating magnetic field may be defined by one or more pulses in one or more pulse trains. The processor of the treatment system 170 may be configured to send signals to a motor threshold device (e.g., to a user interface associated with the motor threshold device) that indicate the timing and/or the power of the pulses of the pulsating magnetic field (e.g., that indicate the timing and power of the drive signals used to generate the pulsating magnetic field).

Although illustrated to include the treatment coil 172 and described primarily with respect to TMS, the treatment system 170 may include any device for administration of therapeutic and/or diagnostic procedure of the human subject. In some examples, the treatment system 170 may be used for a diagnostic procedure (e.g., solely for a diagnostic procedure). Examples of TMS coils are described in are described in U.S. Pat. Nos. 7,824,324, 11,000,693, the contents of which are incorporated herein by reference in their entirety.

The processor (e.g., controller) of the treatment system 170 may be a general purpose processor, a special purpose processor, a conventional processor, a digital signal processor (DSP), a plurality of microprocessors, one or more microprocessors in association with a DSP core, a microcontroller, Application Specific Integrated Circuits (ASICs), Field Programmable Gate Array (FPGAs) circuits, any other type of integrated circuit (IC), a state machine, and the like. The processor may perform signal coding, data processing, power control, input/output processing, and/or any other functionality that enables the treatment system 170 to operate. The processor may be integrated together with one or more other components of the treatment system 170 in an electronic package or chip.

The processor of the treatment system 170 may be coupled to and may receive user input data from and/or output user input data to the treatment coil 172, the articulating arm 174, the display device 176 (e.g., a liquid crystal display (LCD) display unit or organic light-emitting diode (OLED) display unit), and/or the human subject positioning apparatus 182. The processor may access information from, and store data in, any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The processor may access information from, and store data in, memory that is not physically located within the treatment system 170, such as on a server (not shown).

The memory may comprise a computer-readable storage media or machine-readable storage media that maintains computer-executable instructions for performing one or more as described herein. For example, the memory may comprise computer-executable instructions or machine-readable instructions that include one or more portions of the procedures described herein. The processor of the treatment system 170 may access the instructions from memory for being executed to cause the processor to operate as described herein. The memory may comprise computer-executable instructions for executing configuration software. For example, the computer-executable instructions may be executed to perform, in part and/or in their entirety, one or more procedures as described herein. Further, the memory may have stored thereon one or more settings and/or control parameters associated with the treatment system 170, a motor threshold detection device, and/or the user interface for a motor threshold detection device.

The processor may receive power from the power supply, and may be configured to distribute and/or control the power to the other components in the treatment system 170. The power supply may be any suitable device for powering the treatment system 170.

The human subject 180 may be positioned within the human subject positioning apparatus 182. The human subject positioning apparatus 182 may be a chair, recliner, bed, stool, and/or the like. When performing treatment, the treatment coil 172 may be situated such that the human subject's head is positioned under the treatment coil 172. The treatment coil 172 may be adjusted by means of the articulating arm 174 and/or the like. The human subject positioning apparatus 182 may include one or more arms, such as a right arm and a left arm as shown in FIG. 1B. Although not illustrated in FIG. 1B and as described in more detail below, the right and/or left arm of the human subject positioning apparatus 182 may include a motor threshold detection device (not pictured in FIG. 1). The motor threshold detection device may be permanently or removable mounted to the right arm and/or the left arm of the human subject positioning apparatus 182. The motor threshold detection device may be connected to the processor of the treatment system 170, for example, via an electrical connection and/or a wireless connection. In some examples, the motor threshold detection device may include a dedicated display device (not shown), while in other examples, the motor threshold detection device may use the display device 176 of the treatment system 170.

The motor threshold detection device may include one or more movement detection sensors, such as accelerometers, to detect movement of one or more body parts of the human subject 180, such as, but not limited to, fingers, a foot, or the like. The treatment system 170 (e.g., the motor threshold detection device) may be configured to detect movement of the human subject's 180 fingers that is a result from a magnetic pulse generated by the treatment coil 172. For instance, the treatment system 170 may use the motor threshold detection device to detect movement of the fingers of the human subject 180 within a certain time period after a pulse is generated (e.g., movement that occurs within 0.25 second after the generation of the magnetic pulse). The treatment system 170 may ignore movements outside of the time period. It is noted that the examples provided herein correspond to the motor threshold detection device of the treatment system 170 detecting movement of one or more fingers of the human subject 180. However, it should be understood that the motor threshold detection device may detect other bodily movements of the human subject 180 (e.g., a foot of the human subject 180), and examples of detecting bodily movement of the human subject 180 are not limited to only detecting movement of one or more fingers of the human subject 180 (e.g., when treating other disorders, such as OCD and/or PTSD).

The treatment system 170 may comprise one or more computer software applications stored in memory of the treatment system and/or running on the processor. The computer software applications may provide a system graphical user interface (GUI) (e.g., a TMS system GUI) on the display device 176. The computer software applications may incorporate work flow management to guide a technician through the therapeutic and/or diagnostic procedure, and/or supervise and/or control one or more subsystems of the treatment system 170. For example, the computer software applications may control internal system functions, monitor the system status to ensure safe operation, and/or provide the user with a graphical means to manage the preparation for and/or the administration of the therapeutic and/or diagnostic procedure.

Interaction with the computer software applications may be provided via a user interface. In one or more embodiments, the user interface device may be the display device 176, which may be a touch screen display. The display device 176 may include touch activated images of alphanumeric keys and/or buttons for user interaction with the treatment system 170. The display device 176 may provide graphic representations of the system activity, messages, and/or alarms. Interactive buttons, fields, and/or images may be displayed via the display device 176, and may enable the technician to direct and/or interact with system functions, for example, such as entering data, starting and stopping the procedure, running diagnostics, adjusting positioning and/or configuration of the treatment coil 172, adjusting the position of one or more sensor(s), and/or the like.

The treatment system 170 may be used for any therapeutic and/or diagnostic procedure. For example, the treatment system may be used for TMS, transcranial direct current stimulation (tDCS), electroencephalography (EEG), deep brain stimulation (DBS), a diagnostic procedure, and/or the like. For example, the treatment system 170 may be used for any therapeutic and/or diagnostic procedure that includes the placement of electrodes, sensors, probes, and/or the like on a human subject, such as on the surface of a human subject's head. Although described with reference to a head model, the treatment system 170 may be configured to generate a model of any part of the human subject 180, such as, but not limited to, the arm, neck, chest, leg, foot, and/or the like. Example methods of using the treatment system 170 to determine the human subject's 180 MT location and/or treatment location are described in U.S. Pat. Nos. 7,174,947, 9,884,200, the contents of which are incorporated herein by reference in their entirety.

Further, as noted above, prior to TMS treatment, the treatment location of the human subject 180 may first be determined. For TMS, for example, the treatment location is typically determined based on the subject's MT position, which itself is determined by moving the coil near a predicted area determined by patient anatomical landmarks until the desired motor response (e.g., thumb twitch) is achieved. This process may be referred to as a motor threshold detection procedure, and may be part of the TMS treatment procedure (e.g., may be a preliminary step that is performed prior to generating one or more treatment pulse trains). In addition to detecting the location of the MT position, the treatment system 170 may also be used to detect the power of the pulses needed to cause the neurons at the MT location to be depolarized and stimulated (e.g., until the desired motor response in the human subject 180 (e.g., thumb twitch) is achieved). The treatment system 170 may determine the human subject's 180 MT position and power level using the motor threshold detection device (not pictured in FIG. 1).

After the location of the MT position and/or the power level of a test pulse are detected, the MT position and/or power level may be stored. For example, the MT position may be marked, for example, with an ink mark on the subject's head, or skull cap worn on the head but under the coil, or the MT position may be stored within the processor of the treatment system. Further, the power level of the test pulse may be stored, for example, in the memory of the treatment system 170. The treatment location may then be determined using the MT position. Consistent and accurate determination of this MT position leads to consistent and accurate determination of the treatment location. Correctly measuring these precise positions is critical to proper TMS treatment.

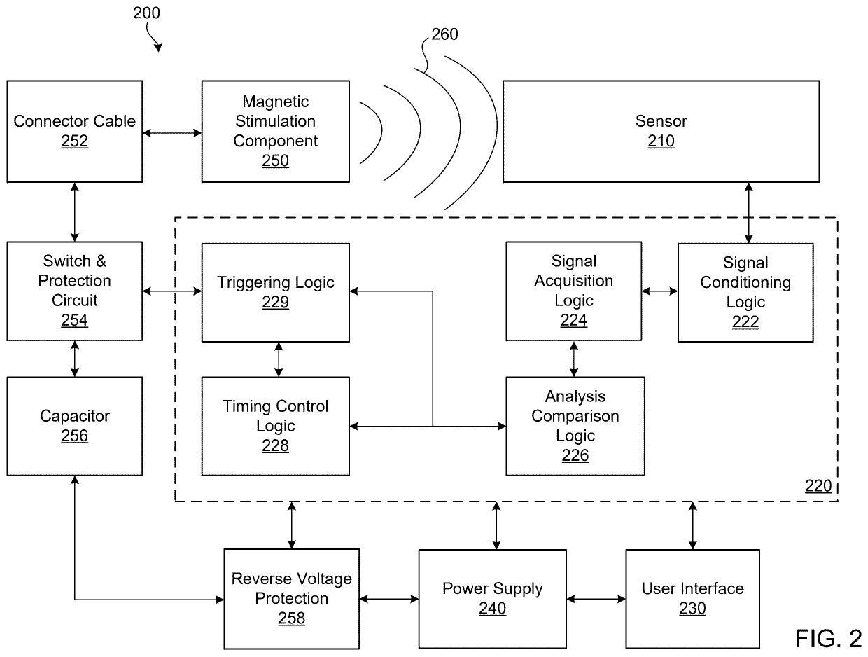

FIG. 2 is a block diagram illustrating an example of a magnetic stimulation system 200. The magnetic stimulation system 200 may comprise a sensor 210, a controller 220, a user interface 230, a power supply 240, a magnetic stimulation component 250, a connector cable 252, a switch and protection circuit 254, a capacitor 256, and reverse voltage protection 258. The magnetic stimulation system 200 may be substantially similar to the magnetic stimulation system 100.

The sensor 210 may be substantially similar to the sensor 110 described herein. The sensor 210 may be configured to perform one or more of the functions described herein with reference to the sensor 110. For example, the sensor 210 may be configured to generate a signal associated with a pulsing magnetic field 260.

The magnetic stimulation component 250 may be substantially similar to the magnetic stimulation component 150 described herein. The magnetic stimulation component 250 may be configured to perform one or more of the functions described herein with reference to the magnetic stimulation component 150. For example, the magnetic stimulation component 250 may generate a pulsing magnetic field 260 that may be used to conduct magnetic stimulation therapy on a treatment area of a patient.

The user interface 230 may be substantially similar to the user interface 130 described herein. For example, the user interface 230 may be configured to perform one or more of the functions described herein with reference to the user interface 130. The power supply 240 may be substantially similar to the power supply 140 described herein. For example, the power supply 240 may be configured to perform one or more of the functions described herein with reference to the power supply 140.

The controller 220 may include signal conditioning logic 222, signal acquisition logic 224, analysis comparison logic 226, triggering logic 229, and timing control logic 228. The controller 220 may be substantially similar to the controller 120 described herein. For example, the controller 220 may be configured to perform one or more of the functions described herein with reference to controller 120. The controller 220 may be any type of hardware, software, and/or combination thereof. The controller 220 may be configured to control one or more of the components of the magnetic stimulation system 200. For example, the controller 220 may be configured to estimate and/or determine one or more characteristics associated with the generated signal, and/or the controller 220 may be configured to determine whether a failure has occurred based on a characteristic of the signal generated by the sensor 210.

The controller 220, for example, via the signal conditioning logic 222, may receive a signal from the sensor 210. The signal conditioning logic 222 may manipulate the signal received from the sensor 210 into a format used by the controller 220 for further processing. For example, the signal conditioning logic 222 may filter, amplify, and/or isolate the signal received from the sensor 210. The signal received from the sensor 210 may include a voltage signal, a current signal, or the like that is proportional to the pulsing magnetic field 260.

The signal acquisition logic 224 may receive the generated signal from the signal conditioning logic 222. The signal acquisition logic 224 may sample the signal and/or convert the signal into a digital signal that may be utilized by the controller 220. The signal acquisition logic 224 may include one or more analog-to-digital converters. For example, the signal acquisition logic 224 may receive an analog signal from the signal conditioning logic 222 and may convert the signal into a digital signal for further processing by the controller 220.

The analysis comparison logic 226 may receive the signal from the signal acquisition logic 224. The analysis comparison logic 226 may compare a characteristic of the signal with a setting of the magnetic stimulation system to determine whether a failure has occurred. For example, the setting of the magnetic stimulation system may include one or more expected characteristics of the pulsing magnetic field 260 generated by the magnetic stimulation component 250. The characteristics of the pulsing magnetic field 260 may be specific to the treatment being conducted by the magnetic stimulation system. As such, the analysis comparison logic 226 may analyze whether the magnetic stimulation system (e.g., the magnetic stimulation component 250) is properly providing treatment.

The timing control logic 228 may coordinate the timing of the components of the magnetic stimulation system 200. For example, the timing control logic 228 may coordinate the timing between the magnetic stimulation component 250 and the signal generated by the sensor 210 (e.g., and received via the controller 220). As such, the timing control logic 228 may ensure that the controller 220 is comparing a signal generated by the sensor 210 with a corresponding pulsing magnetic field 260 generated by the magnetic stimulation component 250.

The triggering logic 229 may initiate actions of the magnetic stimulation system 200 when certain events occur. The triggering logic 229 may receive timing information from the timing control logic 228. The triggering logic 229 may determine the timing relationship between a signal generated by the sensor 210 and the pulsing magnetic field 260. For example, the triggering logic 229 may determine whether or not a signal received from the sensor 210 is in response to the pulsing magnetic field 260. The triggering logic 229 may relay this information to the analysis comparison logic 226 so the occurrence of a failure may be determined.

The controller 220 may include more or less than the components illustrated in FIG. 2. For example, the controller 220 may include an edge detection circuit. The edge detection circuit may be configured to measure a start time and an end time of a pulse of the generated signal indicative of the pulsing magnetic field. The controller 220 may include a peak detection circuit. The peak detection circuit may be configured to receive the generated signal (e.g., from the sensor 210) and determine one or more peaks of a pulse(s) of the generated signal. For example, the peak detection circuit may be configured to determine one or more characteristics (e.g., voltage, current, time, etc.) associated with a peak of a pulse(s) of the generated signal.

The connector cable 252 may provide for an electrical connection and/or electrical communication between the magnetic stimulation component 250 and the switch and protection circuit 254. The switch and protection circuit 254 may be any type of electrical switching device that can operate the magnetic stimulation system (e.g., the magnetic stimulation component 250), for example, by switching power from the capacitor 256 and/or the power supply 240 on and off. For example, the switch and protection circuit 254 may be operated to switch power from the power supply 240 to charge the capacitor 256. The switch and protection circuit 254 may be used to discharge the capacitor 256 through the magnetic stimulation component 250, for example, to generate the pulsing magnetic field 260 that may be used for treatment. As such, when the switch and protection circuit 254 activates to produce the pulse in the magnetic stimulation component 250, currents (e.g., peak currents in excess of 1000 A) may be delivered to the magnetic stimulation component 250 from the charge stored on the capacitor 256.

The capacitor 256 may provide energy storage for the magnetic stimulation component 250. The capacitor 256 may include a single capacitor and/or a capacitor bank. The capacitor 256 may include any number and/or type of capacitor(s) that are appropriate for the power level, charging time, and/or pulse type used by the magnetic stimulation system 200. For example, the capacitor 256 may include eight 10 μF capacitors that may be connected in parallel to result in 80 μF of total capacitance. For example, the capacitor 256 may be a single 80 μF capacitor.

The capacitor 256 may be used, for example, in applications where a 120 VAC power source or the like is available (e.g., where only a 120 VAC power source or the like is available). A typical doctor's office may be equipped with a conventional (e.g., 120 VAC or the like) power supply rather than a higher-power 240 VAC or three-phase power supply. The capacitor 256 may be used to produce higher peak currents in the magnetic stimulation component 250 than would be possible by driving the magnetic stimulation component 250 directly from the power supply 240 alone. For example, the power supply 240 may convert 120 VAC at its input to 1500 VDC at its output, with the DC output capable of producing 1 Amp DC at 1500 VDC. Using capacitor 256 may allow for higher peak pulse current to flow into the magnetic stimulation component 250 than the 1 Amp produced by the power supply 240. As such, the capacitor 256 may be charged up to the power supply's 240 output voltage in the time period between pulses that are delivered to the magnetic stimulation component 250. When the switch and protection circuit 254 activates to produce the pulse in the magnetic stimulation component 250, peak currents in excess of 1000 A may be delivered to the magnetic stimulation component 250 from the charge stored on the capacitor 256. The magnitude of this peak current may be dictated by the inductance of the magnetic stimulation component 250, and/or by the capacitance value and/or voltage level stored on the capacitor 256 prior to the pulse. The capacitor 256 may be used regardless of the type of power supply 240 available. For example, the capacitor 256 may be used in situations where the power supply 240 is a 240 VAC or three-phase power supply to, for example, produce desired peak currents for input into the magnetic stimulation component 250.

The reverse voltage protection 258 may include reverse bias protection for the magnetic stimulation system 200 (e.g., for the magnetic stimulation component). For example, the reverse voltage protection 258 may include one or more diodes (e.g., blocking diode, Schottky diode, etc.) and/or a receiver bias protection switch (e.g., PNP transistor, P-Channel FET, etc.).

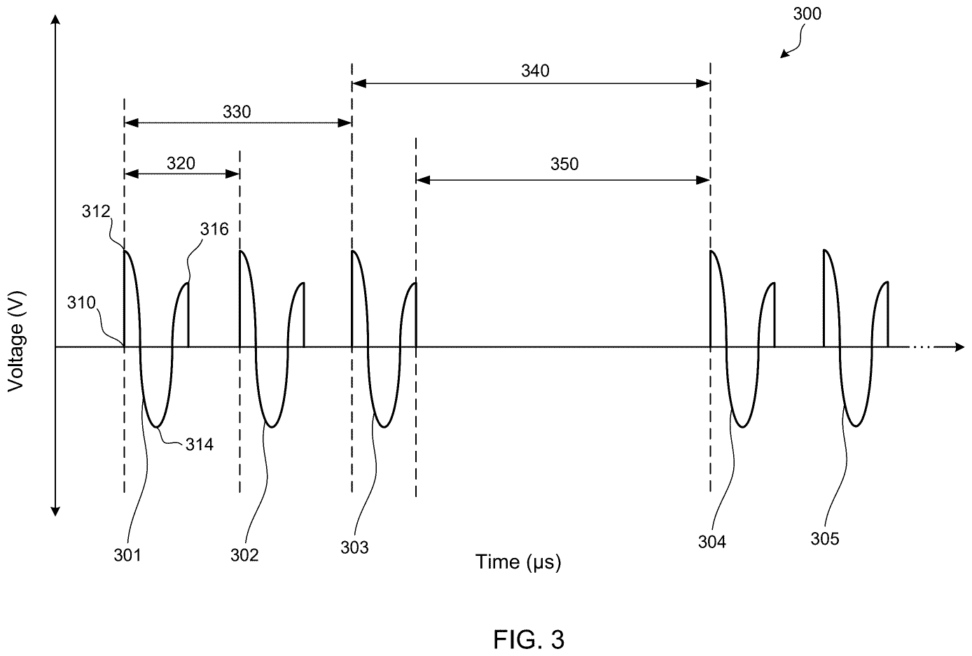

FIG. 3 is a diagram illustrating an example waveform of a signal received by a magnetic stimulation system (e.g., magnetic stimulation system 100 or magnetic stimulation system 200). As described herein, a controller (e.g., controller 120 or controller 220) may receive a signal from a sensor (e.g., sensor 110 or sensor 210) that may be indicative of a pulsing magnetic field (e.g., pulsing magnetic field 160 or pulsing magnetic field 260) generated by a magnetic stimulation component (e.g., magnetic stimulation component 150 or magnetic stimulation component 250). For example, a current may be generated in the sensor that may be proportional to the pulsing magnetic field. The sensor may generate a voltage that may be proportional to a rate of change of the magnetic flux density (dB/dt) of the pulsing magnetic field. The signal received by the controller may include a voltage signal, such as voltage signal 300. Although described with reference to voltage signal 300, the signal received by the controller from the sensor may be in other units (e.g., a current signal, a power signal, and/or the like).

The magnetic stimulation system may be configured to estimate one or more characteristics associated with the signal generated by the sensor (e.g., generated signal 300), for example, as described herein. The magnetic stimulation system may be configured to determine one or more characteristics associated with the generated signal based on the estimated characteristic(s), for example, as described herein. The estimated and/or determined characteristics of the generated signal may include a pulse repetition rate, pulse interval, stimulation interval, timing of a zero-crossing, a timing of a peak of a pulse, amplitude of a peak, pulse shape, peak to RMS ratio, pulse duration, peak duration, rolling average of one or more pulses, and/or the like, for example, as described herein with reference to voltage signal 300.

The voltage signal 300 may include one or more pulses and one or more pulse trains. A pulse of the voltage signal may correspond with a pulse of the pulsing magnetic field. A pulse train may include one or more pulses. The voltage signal 300 may include a first pulse 301, a second pulse 302, a third pulse 303, a fourth pulse 304, and a fifth pulse 305. The first pulse 301, the second pulse 302, and the third pulse 303 may be part of a first pulse train. The fourth pulse 304 and the fifth pulse 305 may be part of a second pulse train.

A pulse may include an initial rising edge, a first peak, a second peak, a third peak, and a pulse interval. For example, the first pulse 301 may include an initial rising edge 310, a first peak 312, a second peak 314, a third peak 316, and a pulse interval 320. The initial rising edge of a pulse may be a time when the pulse begins, for example, when the pulse exceeds 0 V. The first peak of the pulse may be characterized by a time and a maximum voltage of the pulse after the initial rising edge and before the pulse decreases back to 0 V. The second peak of the pulse may be characterized by a time and a minimum voltage of the pulse after a zero-crossing after the first peak and before the pulse increases back to 0 V. The third peak of the pulse may be characterized by a time and a second maximum voltage after a zero-crossing after the second peak and before the pulse decreases back to 0 V. After the third peak, the pulse may decrease to 0 V (e.g., after crossing 0 V one or more times, for example, oscillating) and before an initial rising edge of a subsequent pulse.

The pulse interval may be indicative of a time between an initial rising edge of a pulse to an initial rising edge of a subsequent pulse. For example, the pulse interval 320 may be indicative of the time between the initial rising edge 310 of the first pulse 301 to the initial rising edge of the second pulse 302. The stimulation time of a pulse train may be calculated based on the time between the initial rising edge of a first pulse in the pulse train to the initial rising edge of the last pulse in the pulse train. For example, stimulation time 330 may indicate the time duration between the initial rising edge 310 of the first pulse 301 to the initial rising edge of the third pulse 303. For example, the stimulation time 330 may be indicative of the duration of time of a pulse train of the pulsing magnetic field. The stimulation interval may be indicative of a time between the initial rising edge (e.g., the first peak) of a last pulse in a pulse train to the initial rising edge (e.g., the first peak) of a first pulse in a subsequent pulse train. For example, the stimulation interval 340 may be indicative of the time between pulse trains of a pulsing magnetic field. For example, the stimulation interval 340 may indicate the time between the initial rising edge (e.g., the first peak) of the third pulse 303 (e.g., which may be the last pulse of the first pulse train) to the initial rising edge (e.g., the first peak) of the fourth pulse 304 (e.g., which may be the first pulse of the second pulse train). Inter-train interval 350 may indicate the time duration between the last pulse of a first pulse train (e.g., the third pulse 303) and the initial rising edge of the first pulse of a second (e.g., successive) pulse train (e.g., the fourth pulse 304).

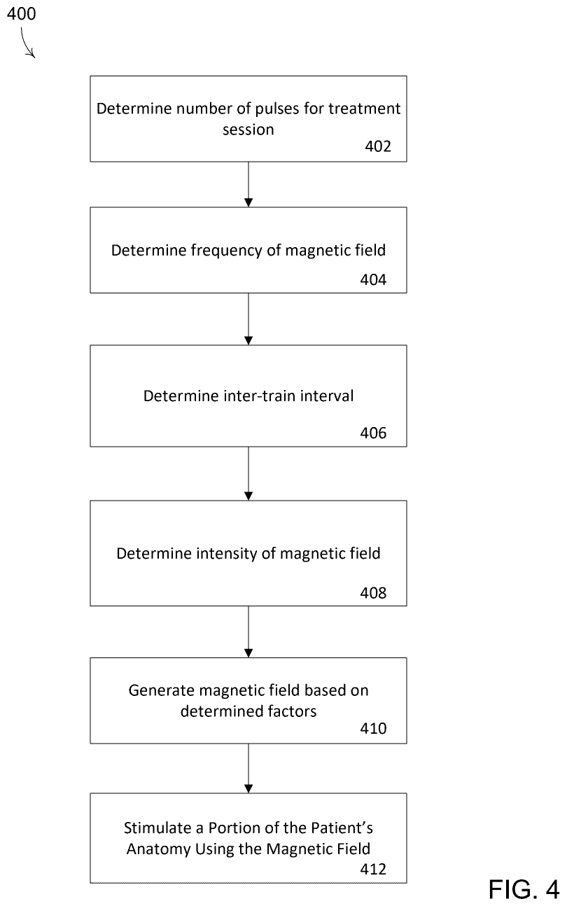

FIG. 4 is a flow diagram of an example TMS treatment process 400. The TMS treatment process 400 may be performed using a magnetic stimulation system (e.g., magnetic stimulation system 100 or magnetic stimulation system 200). The TMS treatment process may be used to determine one or more parameters for a treatment session of a plurality of treatment sessions that may be administered to one or more patients.

At 402, a number of pulses of a pulsing magnetic field for a treatment session may be determined. For example, the number of pulses of the pulsing magnetic field for the treatment session may be within the range of 1,700-6,000 pulses per session. In a further example, the number of pulses may be within the range of 2,800-3,200 pulses per session. In yet a further example, the number of pulses may be within the range of 2,900 to 3,100 pulses per session. Each pulse may be generated using the signals described in FIG. 3.

Referring again to FIG. 4, the pulses of the pulsing magnetic field for the treatment session may be sub-divided into one or more pulse trains (e.g., as described herein). For example, each pulse train may comprise between 15 and 120 pulses. The total number of pulse trains administered during a given treatment session may depend on the total number of pulses and the number of pulses per pulse train. For example, each treatment session may comprise between 30 and 120 pulse trains. In another example, each treatment session may comprise between 50 and 100 pulse trains. In yet another example, each treatment session may comprise between 70 and 90 pulse trains. Different treatment sessions may comprise different numbers of pulses, different numbers of pulse trains, and/or different numbers of pulses per pulse train.

Additionally, the duration of a given pulse train may depend on the frequency of the pulsing magnetic field and a number of pulses per pulse train. For example, a duration of a given pulse train may be between 1 second and 12 seconds. In another example, the duration of a given pulse train may be between 4 second and 8 seconds. As different pulse trains may comprise different numbers of pulses or different frequencies, different pulse trains may therefore have different durations.

At 404, a frequency of the pulsing magnetic field for the treatment session may be determined. The frequency of the pulsing magnetic field may represent a number of pulses of the pulsing magnetic field per second (e.g., or other unit of time), in which case the frequency may be measured in units of Hertz (Hz). For example, the frequency of the pulsing magnetic field for the treatment session may be between 5 and 20 Hz. In another example, the frequency of the pulsing magnetic field for the treatment session may be between 5 and 15 Hz. In yet another example, the frequency of the pulsing magnetic field for the treatment session may be approximately 10 Hz. Different treatment sessions may comprise different frequencies of the pulsing magnetic field.

At 406, an inter-train interval for the treatment session may be determined. The inter-train interval may represent an amount of time between successive pulse trains. For example, the inter-train interval may be between 11 and 14 seconds. In another example, the inter-train interval may be approximately 11 seconds.

At 408, an intensity of the magnetic field may be determined. For example, the intensity of the magnetic field may be determined using one or more methods described herein. The intensity of the magnetic field may be determined relative to, for example, an intensity required to provoke a motor threshold (MT) response in a given patient. For example, the intensity of the pulsing magnetic field may be between 50% and 120% of the intensity required to provoke the MT response in the patient. In another example, the intensity of the pulsing magnetic field may be between 110% and 120% of the intensity required to provoke the MT response in the patient. In a further example, the intensity of the pulsing magnetic field may be approximately 120% of the intensity required to provoke the MT response in the patient.

In order to determine the intensity of the pulsing magnetic field for the treatment session, the intensity required to provoke the MT response in a patient may first be determined, and then the intensity of the pulsing magnetic field for the treatment session may be calculated based on the intensity required to provoke the MT response. For example, the intensity required to provoke the MT response in the patient may be determined by determining a reference point (e.g., a reference or MT location) on the patient, determining a first intensity for a first pulse of the pulsing magnetic field (e.g., based on one or more factors associated with the patient), generating the first pulse of the pulsing magnetic field at the MT location, and determining whether the first pulse provoked the MT response in the patient. If the first pulse provoked the MT response, the intensity of the first pulse may be set as the intensity required to provoke the MT response. Alternatively, if the first pulse did not provoke the MT response, the process may be repeated with successive pulses of increasing intensity until a pulse provokes the MT response. The MT response may be, for example, a thumb or foot twitch.

Determining the intensity of the pulsing magnetic field for the treatment session may include determining the MT location of the patient. The magnetic stimulation system may be configured to generate a magnetic field in the subject's brain that stimulates a strip shaped region of the subject's brain (e.g., in an anterior-posterior direction) that encompasses the motor threshold location. The intensity of the magnetic field needed to generate the MT response at the MT location may be used to determine the intensity of the pulsing magnetic field for the treatment session.

Alternatively, the patient may be a repeat patient for whom the intensity required to provoke the MT response was previously determined. In this case, the intensity required to provoke the MT response may be determined by determining an identity of the patient and accessing a database that includes associations between respective patient identities and respective magnetic field intensities required to provoke the MT response in the patient.

At 410, the magnetic field may be generated based on the determined parameters. For example, the magnetic stimulation system may generate one or more signals, which the controller (e.g., the controller 120 and/or the controller 220) may use to generate the pulsing magnetic field. The magnetic field may then be applied to a treatment location on the patient, which may be determined using one or more of the methods described herein. For example, the magnetic field generated by the magnetic stimulation system may be used to stimulate one or more portions of the patient's anatomy (e.g., one or more portions of the patient's brain) at 412 to perform the treatment session.

For example, the frequency of the pulsing magnetic field may be between approximately 10 and 25 Hz. The number of pulses per session may be between approximately 2000 and 5000 pulses. A patient may receive a total number of sessions that is between approximately 20 and 50 sessions. The total number of sessions may be split over a number of (e.g., consecutive) days such that the total number of sessions per day is between approximately 2 and 10 sessions. Sessions performed within the same day may be administered regularly (e.g., at the same time every hour for a space of two or more hours), and the amount of time between the end of a first session and the beginning of a second session (e.g., the inter-session interval) may be dependent on the frequency, the total number of pulses per session, and the inter-train interval. The inter-session interval may be between approximately 0 and 43.75 minutes. The inter-train interval may be between approximately 11 and 14 seconds, and the number of pulses may be between approximately 2000 and 4000 pulses per session. The inter-train interval may be less than 25 seconds, and five or more sessions may be performed per day.

In a first example, one or more treatment sessions may be performed with approximately 3000 pulses per session (e.g., which may be divided into approximately 75 pulse trains), at an intensity of 120% of the intensity required to provoke an MT response in the patient and a frequency of 10 Hz, with a 4-second train duration, and an 11 second inter-train interval. In this example, there may be approximately 36 sessions administered to the patient over the course of 5 days (e.g., with approximately 6-8 sessions per day). The sessions may be performed at the same time every hour for a space of 6-8 hours, with an inter-session interval of approximately 41 minutes. The inter-session interval may depend on the frequency of performing sessions and the length of each session.

In a second example, one or more treatment sessions may be performed with approximately 5000 pulses per session (e.g., which may be divided into approximately one or more pulse trains), at an intensity of 120% of the intensity required to provoke an MT response in the patient and a frequency of 20 Hz, with a 2-second train duration, and an 11 second inter-train interval. In this example, there may be approximately 50 sessions administered to the patient over the course of 5 days (e.g., with approximately 10 sessions per day). The sessions may be performed at the same time every hour for a space of 10 hours, with an inter-session interval of approximately 33 minutes. The inter-session interval may depend on the frequency of performing sessions and the length of each session.

In a third example, one or more treatment sessions may be performed with approximately 4000 pulses per session (e.g., which may be divided into approximately one or more pulse trains), at an intensity of 120% of the intensity required to provoke an MT response in the patient and a frequency of 15 Hz, with a 3-second train duration, and an 11 second inter-train interval. In this example, there may be approximately 40 sessions administered to the patient over the course of 5 days (e.g., with approximately 8 sessions per day). The sessions may be performed at the same time every hour for a space of 8 hours, with an inter-session interval of approximately 37 minutes. The inter-session interval may depend on the frequency of performing sessions and the length of each session.

Although FIG. 4 describes determining magnetic stimulation parameters for a single treatment session, it should be appreciated that multiple treatment sessions may be administered to the same patient, for example as part of an accelerated DASH protocol. Successive treatment sessions may be separated by an inter-session interval, during which a magnetic field may not be administered to the patient. The inter-session interval may vary, but may be such that multiple treatment sessions are performed within the same day.

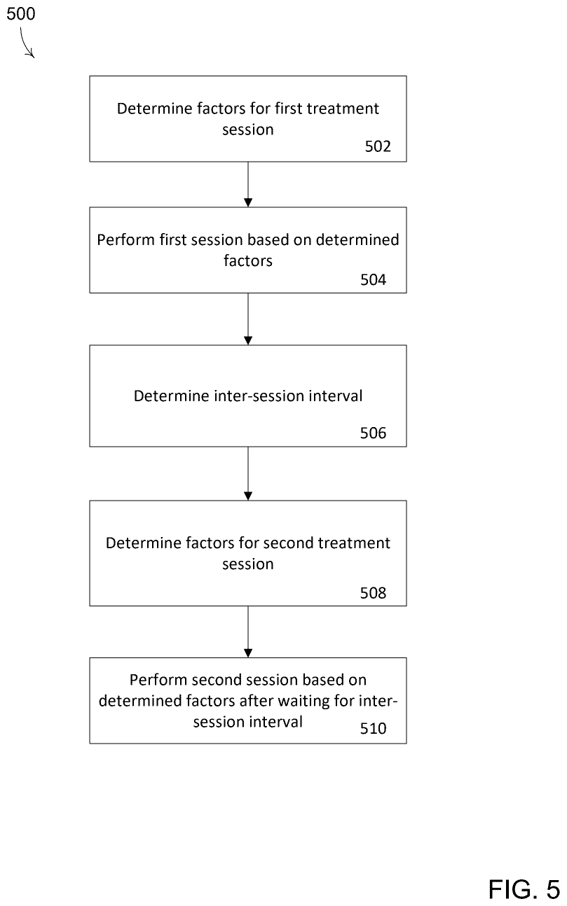

FIG. 5 is a flow diagram of an example TMS treatment process 500 according to an accelerated DASH protocol. The TMS treatment process 500 may be performed using a magnetic stimulation system (e.g., magnetic stimulation system 100 or magnetic stimulation system 200). The TMS treatment process may be used to determine one or more parameters for multiple treatment sessions, as well as an inter-session interval between respective treatment sessions.

At 502, one or more parameters may be determined for a first treatment session of a plurality of treatment sessions. The parameters may apply to treatment sessions applied to one or more patients. For example, the parameters for the first treatment session may include a number of pulses for the first treatment session (e.g., which may be determined as described at 402 of FIG. 4), a frequency of the magnetic field for the first treatment session (e.g., which may be determined as described at 404 of FIG. 4), an inter-train interval associated with the first treatment session (e.g., which may be determined as described at 406 of FIG. 4), and/or an intensity of the magnetic field for the first treatment session (e.g., which may be determined as described at 408 of FIG. 4).

At 504, the first treatment session may be performed on a patient based on the parameters determined at 502. At 506, an inter-session interval may be determined. The inter-session interval may be determined before the first treatment session is performed. The inter-session interval may represent an amount of time between successive (e.g., consecutive) treatment sessions. For example, the inter-session interval may be between 5 and 240 minutes (e.g., between 5 minutes and 4 hours). In another example, the inter-session interval may be between 15 and 90 minutes. In a further example, the inter-session interval may be between 30 and 75 minutes. In yet another example, the inter-session interval may be between 45 and 60 minutes. During the inter-session interval, no magnetic field may be applied to the patient. Additionally, it should be appreciated that the inter-session interval may not be fixed, and a first inter-session interval between a first treatment session and a second treatment session may be different from or the same as a second inter-session interval between the second treatment session and a third treatment session. For example, the first inter-session interval may be approximately 60 minutes, while the second inter-session interval may be approximately 45 minutes. An earlier inter-session interval may be longer than, shorter than, or the same length as a later inter-session interval.

At 508, one or more parameters may be determined for a second treatment session of the plurality of treatment sessions. For example, the parameters for the first treatment session may include a number of pulses for the second treatment session (e.g., which may be determined as described at 402 of FIG. 4), a frequency of the magnetic field for the second treatment session (e.g., which may be determined as described at 404 of FIG. 4), an inter-train interval associated with the second treatment session (e.g., which may be determined as described at 406 of FIG. 4), and/or an intensity of the magnetic field for the second treatment session (e.g., which may be determined as described at 408 of FIG. 4). At 510, the second treatment session may be performed on the patient based on the parameters determined at 508. It should be noted that further treatment sessions may be performed on the patient based on determined parameters and/or inter-session interval(s). The first treatment session and the second treatment session (e.g., and one or more further treatment sessions) may be performed within the same day.

Each of the determined parameters for the second treatment session may be the same as or different from the determined parameters for the first treatment session. For example, the second treatment session may have a lower number of pulses as compared to the first treatment session, a greater number of pulses as compared to the first treatment session, or the same number of pulses as the first treatment session. For example, the number of pulses for the second treatment session may be approximately 80-99% of the number of pulses for the first treatment session. The second treatment session may have a greater frequency of the magnetic field as compared to the first treatment session, a lower frequency of the magnetic field as compared to the first treatment session, or the same frequency of the magnetic field as the first treatment session. The second treatment session may have a greater inter-train interval as compared to the first treatment session, a lower inter-train interval as compared to the first treatment session, or the same inter-train interval as the first treatment session. The second treatment session may have a greater intensity of the magnetic field compared to the first treatment session, a lower intensity of the magnetic field compared to the first treatment session, or the same intensity of the magnetic field as the first treatment session. For example, increasing the intensity of the magnetic field (e.g., the amplitude of the treatment session) across successive treatment session may allow for easing the patient into the full treatment level.

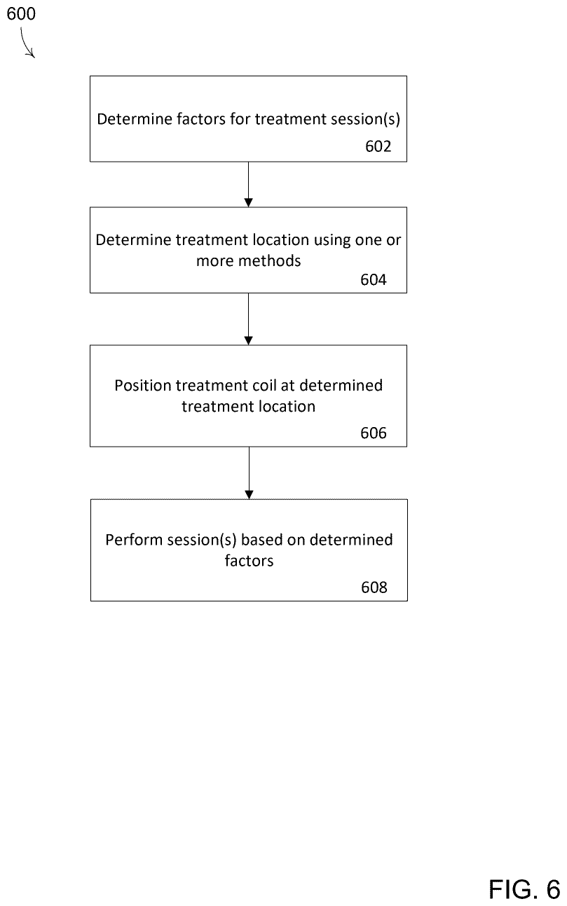

FIG. 6 is a flow diagram of an example method 600 for determining a treatment location for a TMS treatment process. The method 600 may be performed using a magnetic stimulation system (e.g., magnetic stimulation system 100 or magnetic stimulation system 200). The method may be used to determine a treatment location for one or more treatment sessions, as well as one or more parameters for the treatment sessions.

At 602, one or more parameters for the treatment session(s) may be determined. For example, the parameters for the first treatment session may include a number of pulses for the treatment session(s) (e.g., which may be determined as described at 402 of FIG. 4), a frequency of the magnetic field for the treatment session(s) (e.g., which may be determined as described at 404 of FIG. 4), an inter-train interval associated with the treatment session(s) (e.g., which may be determined as described at 406 of FIG. 4), and/or an intensity of the magnetic field for the treatment session(s) (e.g., which may be determined as described at 408 of FIG. 4). As described herein, there may be multiple treatment sessions, and one or more of the parameters may be the same or different across different treatment sessions. Further, an inter-session interval may be determined (e.g., as described at 506 of FIG. 5). If there are more than two treatment sessions (e.g., more than one inter-session intervals), the inter-session intervals may be the same or different.

At 604, a treatment location may be determined using one or more methods. For example, the treatment location may be located on the patient's head, and may be determined based on a size of the patient's head, a portion of the patient's brain that is to be treated, and/or the like. The treatment location may be a left dorsolateral prefrontal cortex (DLPFC) of the patient. The treatment location may be determined with magnetic resonance imaging (MRI) data, or without requiring the use of MRI data.

In a first example, the treatment location may be determined based on the location of a motor threshold (MT) location on the patient's head. For example, a magnetic stimulation system (e.g., a TMS device) may be configured to generate a magnetic field in the subject's brain that stimulates a strip shaped region of the subject's brain (e.g., in an anterior-posterior direction) that encompasses the MT location. The MT location may be determined using localization of the TMS device. For example, the TMS device may be moved over an area of the subject's head until an indication of positioning is observed (e.g., until the subject's thumb moves or twitches indicating a motor threshold location). The MT location may be determined, for example, using a stimulation frequency rate of approximately one (1) Hz. From the MT location, the TMS device may be moved to the desired treatment location on the subject. In an example, the desired treatment location may be a predefined distance from the determined MT location. For example, the predefined distance may be between 4.0 and 7.0 cm anteriorly from the determined MT location. In another example, the predefined distance may be between 5.0 and 6.0 cm anteriorly from the determined MT location. In yet another example, the predefined distance may be approximately five centimeters (5 cm) anteriorly from the determined MT location.

In another example, the treatment location may be determined based on a head support guide. In this example, a location of a reference point defined by a support structure supported by a head of the patient may be determined. A position angle associated with the treatment location relative to the reference point may then be determined. The position angle may represent an angular offset between a line located within a median plane of the patient that intersects with the reference point and a line that interests the reference point and the MT location. A distance between the support structure and the treatment location (e.g., an arc length) may then be determined. Finally, the treatment location may be determined based on the distance between the support structure and the treatment location, and the position angle. The treatment location may be determined based on one or more structural or functional features in a medical image (e.g., an MRI image), or without requiring the use of medical imaging data (e.g., MRI data). The treatment location may be a first treatment location, and a second treatment location may be determined (e.g., in addition to the first treatment location.

In yet another example, the treatment location may be determined based on measuring a head circumference of the patient, a nasion-inion distance of the patient, and a head length of the patient. One or more standardized coordinates associated with the treatment location may be determined, and the treatment location may be calculated relative to a surface reference location (e.g., or locations) based on the head circumference, the nasion-inion distance, the head length, and the standardized coordinates. The standardized coordinates may represent group average coordinates on a standard (e.g., MNI) brain that reflect the location of a given brain structure. The surface reference location may comprise Brodmann area 46. For example, one or more target coordinates may be transformed to device coordinates. The target coordinates may represent coordinates on the patient's brain (e.g., at the treatment location), and the device coordinates may represent coordinates associated with a device used to determine the treatment location. A distance of the treatment location from the surface reference location(s) may be estimated based on the head circumference, the nasion-inion distance, and/or the head length. For example, the distance may be estimated based on a ration of a surface distance to the head length. An angle relative to one or more of a head feature or a device feature may be determined based on the transformed device coordinates and/or the head circumference, the nasion-inion distance, and the head length. For example, the angle may be determined based on the ratio of the surface distance to the head length. One or more additional measurements may be made. For example, an ear-to-ear distance may be measured, and the distance may be estimated based on a ration of the ear-to-ear distance to a measured head width.

In yet another example, the treatment location may be determined based on measuring the head circumference of the patient, the ear-to-ear distance of the patient, and the head width of the patient. One or more standardized coordinates associated with the treatment location may then be determined. The treatment location may be calculated relative to a surface reference location(s) (e.g., Brodmann area 46) based on the head circumference, the ear-to-ear distance, the head width, and the standardized coordinates.

In yet another example, the treatment location may be determined based on or more of an EEG of the patient, a heart rate measurement of the patient, and/or a blood oxygenation measurement of the patient.

Once the treatment location is determined at 604, the magnetic stimulation component 150 (e.g., the treatment coil) may be positioned at the determined treatment location at 606, and the one or more sessions may be performed based on the determined parameters at 608. For example, the treatment session(s) may be performed as described with reference to 412 of FIG. 4, and/or 504 and/or 510 of FIG. 5. For example, if multiple treatment sessions are to be performed, each treatment session may have the same or different parameters, and there may be varying inter-session intervals between each treatment session.

TMS devices, such as the example magnetic stimulation systems described herein, may be used to treat a number of conditions or disorders, for example depression, incontinence, and weight control issues. Such treatments may be applied to a subject, for example, using the example TMS devices in accordance with the example TMS treatment process 400 or the example TMS treatment process 500. The example TMS devices may be used to treat other conditions or disorders. For example, the TMS devices may be used in the rehabilitation of muscles. The TMS devices may be used in the treatment of peripheral nervous system disorders.

The example TMS devices may be used in one or more of the following treatment contexts, including major depressive disorder, epilepsy, schizophrenia, Parkinson's disease, Tourette's syndrome, amyotrophic lateral sclerosis (ALS), multiple sclerosis (MS), Alzheimer's disease, attention deficit/hyperactivity disorder (ADHD), obesity, bipolar disorder and/or mania, anxiety disorders (e.g., panic disorder with and without agoraphobia, social anxiety disorder, acute stress disorder, generalized anxiety disorder), post-traumatic stress disorder (PTSD), obsessive compulsive disorder (OCD), pain (e.g., migraine, trigeminal neuralgia), chronic pain disorders (e.g., pain due to diabetic neuropathy, post-herpetic neuralgia), idiopathic pain disorders (e.g., fibromyalgia, regional myofascial pain syndrome), rehabilitation following stroke (neuro plasticity induction), tinnitus, stimulation of implanted neurons to facilitate integration, substance-related disorders (e.g., dependence, abuse, and/or withdrawal diagnoses for alcohol, cocaine, amphetamine, caffeine, nicotine, cannabis, etc.), spinal cord injury and regeneration and/or rehabilitation, head injury, sleep deprivation reversal, primary sleep disorders (e.g., primary insomnia, primary hypersomnia, or circadian rhythm sleep disorder), cognitive enhancements, dementias, premenstrual dysphoric disorder (PMS), drug delivery systems (e.g., changing cell membrane permeability to a drug), induction of protein synthesis (e.g., induction of transcription and translation), stuttering, aphasia, dysphagia, essential tremor, and eating disorders (e.g., bulimia, anorexia, binge eating).

It should be appreciated that the example TMS devices may be employed for uses other than treatment applications. For example, the example TMS devices may be used (e.g., in accordance with the example TMS treatment processes 400 and/or 500) to perform diagnoses of one or more conditions in a subject. To illustrate, the example TMS devices may be used to diagnose a subject's response to drugs or other therapies, and/or may be used to quantify an effectiveness of such therapies. For example, a pharmaceutical may be known to have effects (e.g., direct or secondary effects) on the performance of the central nervous system. Such effects may be observed using the example TMS devices, for example by providing TMS and observing one or more of evoked potentials, motor response, conduction velocities, or other responses. Observed changes in one or more such response may be used, for example, to quantify a performance of the pharmaceutical or to determine an optimal dosing of the pharmaceutical.

The example TMS devices may be used (e.g., in accordance with the example TMS treatment processes 400 and/or 500) to perform diagnoses of one or more pathologies in a subject, for example by observing neurological response. Such pathologies may include, but are not limited to, degenerative diseases, extent of a traumatic injury, progression of a disease, systemic deficiencies, and congenital anomalies. To illustrate, the example TMS devices may be used in the diagnosis of, for example, compromised motor function, Alzheimer's disease, Parkinson's disease, ALS, MS, diabetic neuropathy, chronic demyelinating neuropathy, acute demyelinating neuropathy, epilepsy, vitamin B12 deficiency (e.g., pernicious anemia), vitamin E deficiency, neurosarcoidosis, tinnitus, and stroke. The example TMS devices may be used to evaluate the efficacy of treatments for such pathologies. For example, the TMS devices may be used to assess and/or measure the effect of pharmaceuticals, for example anti-convulsives, Alzheimer's medications, anti-psychotics, pain medications, antianxiety medications, hypnotics (sedatives), analgesics (central), ADHD medications, or anesthetics.

It should be appreciated that the example TMS devices described herein are not limited to their illustrated configurations. For example, one or more components from a first one of the example TMS devices may be implemented in a second one of the example TMS devices. One of ordinary skill in the art will appreciate that these and other different configurations of the example Magnetic stimulation systems may be implemented without departing from the scope and spirit of the instant disclosure.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

What is claimed is:

1. A method of generating a pulsing magnetic field related to magnetic stimulation therapy of a patient, the method comprising:
generating a plurality of signals associated with the generation of the pulsing magnetic field for a plurality of treatment sessions, wherein generating the plurality of signals for each treatment session of the plurality of treatment sessions comprises:
determining a number of pulses of the pulsing magnetic field for the treatment session; wherein the number of pulses of the pulsing magnetic field for the treatment session is within a range of 2000-4000 pulses;
determining a frequency of the pulsing magnetic field for the treatment session;
determining an inter-train interval of the pulsing magnetic field for the treatment session, wherein the inter-train interval of the pulsing magnetic field for the treatment session is within a range of 11 to 14 seconds; and
determining an intensity of the pulsing magnetic field for the treatment session; and
generating the pulsing magnetic field for the magnetic stimulation therapy in response to the signals based on the determined number of pulses, frequency, inter-train interval, and intensity of the pulsing magnetic field for each treatment session of the plurality of treatment sessions;
wherein an amount of time between consecutive treatment sessions of the plurality of treatment sessions is within a range of 5 to 240 minutes.

2. The method of claim 1, wherein the number of pulses of the pulsing magnetic field for each treatment session of the plurality of treatment sessions is within a range of 2,800 to 3,200 pulses.

3. The method of claim 1, wherein the number of pulses of the pulsing magnetic field for each treatment session of the plurality of treatment sessions is within a range of 2,900 to 3,100 pulses.

4. The method of claim 1, wherein the number of pulses of the pulsing magnetic field for each treatment session of the plurality of treatment sessions is approximately 3000 pulses.

5. The method of claim 1, wherein the frequency of the pulsing magnetic field for each treatment session of the plurality of treatment sessions is within a range of 5 to 20 Hz.

6. The method of claim 1, wherein the frequency of the pulsing magnetic field for each treatment session of the plurality of treatment sessions is within a range of 5 to 15 Hz.

7. The method of claim 1, wherein the frequency of the pulsing magnetic field for each treatment session of the plurality of treatment sessions is approximately 10 Hz.

8. The method of claim 1, wherein the inter-train interval of the pulsing magnetic field for each treatment session of the plurality of treatment sessions is 11-12 seconds.

9. The method of claim 1, wherein determining an intensity of the pulsing magnetic field for each treatment session of the plurality of treatment sessions comprises:
determining an intensity required to provoke a motor threshold (MT) response in the patient; and
calculating the intensity of the pulsing magnetic field for each treatment session of the plurality of treatment sessions based on the intensity required to provoke the MT response in the patient.

10. The method of claim 9, wherein the intensity of the pulsing magnetic field for each treatment session of the plurality of treatment sessions is within a range of 50% to 120% of the intensity required to provoke the MT response in the patient.

11. The method of claim 9, wherein the intensity of the pulsing magnetic field for each treatment session of the plurality of treatment sessions is within a range of 110% to 120% of the intensity required to provoke the MT response in the patient.

12. The method of claim 9, wherein the intensity of the pulsing magnetic field for each treatment session of the plurality of treatment sessions is approximately 120% of the intensity required to provoke the MT response in the patient.

13. (canceled)

14. (canceled)

15. (canceled)

16. The method of claim 1, further comprising:
determining a location of an MT location on the patient;
measuring a predetermined distance from the MT location, wherein the predetermined distance is within a range of 4.0 and 7.0 cm; and
determining a treatment location based on measuring the predetermined distance from the MT location.

17. The method of claim 16, wherein the predetermined distance is within a range of 5.0 and 6.0 cm.

18-24. (canceled)

25. The method of claim 1, wherein the plurality of treatment sessions comprises a first treatment session and a second treatment session, and wherein the first treatment session and the second treatment session are administered to the patient on a same day.

26. (canceled)

27. The method of claim 1, wherein the amount of time between consecutive treatment sessions of the plurality of treatment sessions is within a range of 15 to 90 minutes.

28. The method of claim 1, wherein the amount of time between consecutive treatment sessions of the plurality of treatment sessions is within a range of 30 to 75 minutes.

29. The method of claim 1, wherein the amount of time between consecutive treatment sessions of the plurality of treatment sessions is within a range of 45 to 60 minutes.

30. The method of claim 25, wherein a number of pulses for the first treatment session is different from a number of pulses for the second treatment session.

31. (canceled)

32. (canceled)

33. (canceled)

34. The method of claim 1, further comprising subdividing the number of pulses for each treatment session of the plurality of treatment sessions into one or more pulse trains, wherein the inter-train interval comprises an amount of time between generating respective pulse trains.

35. The method of claim 34, wherein each pulse train of the one or more pulse trains comprises between 15 and 120 pulses.

36. The method of claim 34, wherein each treatment session of the plurality of treatment sessions comprises between 30 and 120 pulse trains.

37. The method of claim 34, wherein each treatment session of the plurality of treatment sessions comprises between 50 and 100 pulse trains.

38. The method of claim 34, wherein each treatment session of the plurality of treatment sessions comprises between 70 and 90 pulse trains.

39. The method of claim 34, wherein a duration of each pulse train of the one or more pulse trains is between 1 second and 12 seconds.

40. The method of claim 34, wherein a duration of each pulse train of the one or more pulse trains is between 4 seconds and 8 seconds.

41. The method of claim 1, further comprising:
measuring a head circumference of the patient, a nasion-inion distance of the patient, and a head length of the patient;
determining one or more standardized coordinates associated with the treatment location; and
calculating a treatment location relative to a surface refence location based on the head circumference, the nasion-inion distance, the head length, and the standardized coordinates.

42. The method of claim 41, wherein calculating the treatment location based on the head circumference, the nasion-inion distance, the head length, and the standardized coordinates comprises:
transforming one or more target coordinates to device coordinates;
estimating a distance of the treatment location from the surface reference location based on the head circumference, the nasion-inion distance, and the head length; and
determining an angle relative to one or more of a head feature or a device feature based on the transformed coordinates and the head circumference, the nasion-inion distance, and the head length.

43. The method of claim 42, where the distance is estimated based on a ratio of a surface distance to the head length.

44. The method of claim 43, wherein the angle is determined based on the ratio of the surface distance to the head length.

45. (canceled)

46. (canceled)

47. (canceled)

48. The method of claim 42, wherein the surface reference location comprises Brodmann area 47.

49. The method of claim 1, further comprising:
measuring a head circumference of the patient, an ear to ear distance of the patient, and a head width of the patient;
determining one or more standardized coordinates associated with the treatment location; and
calculating a treatment location relative to a surface refence location based on the head circumference, the ear to ear distance, the head width, and the standardized coordinates.

50. (canceled)

51. The method of claim 1, wherein the inter-train interval of the pulsing magnetic field for each treatment session of the plurality of treatment sessions is approximately 11 seconds.
